(19) United States
(12) Patent Application Publication
(10) Pub. No.: US 2026/0146236 A1
(43) Pub. Date: May 28, 2026

(54) HUMAN SEBACEOUS GLAND CARCINOMA CELL LINE AND US THEREOF

(71) Applicant: SHANGHAI NINTH PEOPLE'S HOSPITAL, SHANGHAI JIAOTONG UNIVERSITY SCHOOL OF MEDICIN, Shanghai (CN)

(72) Inventors: Xiang GU, Shanghai (CN); Shiqiong Xu, Shanghai (CN); Xianqun Fan, Shanghai (CN); Renbing Jia, Shanghai (CN); Ziyue Huang, Shanghai (CN); Hao Tian, Shanghai (CN)

(73) Assignee: SHANGHAI NINTH PEOPLE'S HOSPITAL, SHANGHAI JIAOTONG UNIVERSITY SCHOOL OF MEDICIN, SHANGHAI NINTH PEOPLE'S HOSPITAL, SHANGH (CN)

(21) Appl. No.: 19/449,479

(22) Filed: Jan. 15, 2026

Related U.S. Application Data
(63) Continuation of application No. PCT/CN2024/108119, filed on Jul. 29, 2024.

(30) Foreign Application Priority Data
Sep. 12, 2023 (CN) 202311176126.X

(51) Int. Cl. C12N 5/09; A01K 67/0271; G01N 33/50

(52) U.S. Cl. CPC C12N 5/0693; A01K 67/0271; G01N 33/5011; A01K 2207/12; A01K 2207/15; A01K 2267/0331; C12N 2503/00; C12N 2503/02

(57) ABSTRACT

A human sebaceous gland carcinoma cell line and use thereof are provided. The human sebaceous gland carcinoma cell line SHNPH-SeC was deposited in the China Center for Type Culture Collection (CCTCC) on Aug. 31, 2023, with an accession number CCTCC NO: C2023113. The human sebaceous gland carcinoma cell line SHNPH-SeC carries a TP53 mutation, and has been identified as a novel single cell line by short tandem repeat (STR) genotyping. This human sebaceous gland carcinoma cell line exhibits strong adherence and stable cellular characteristics, can be stably passaged for multiple generations, and possesses rapid proliferation and migration capabilities, which are in line with the characteristics of malignant tumor cells.

### CROSS REFERENCE TO THE RELATED APPLICATIONS

This application is a continuation application of International Application No. PCT/CN2024/108119, filed on Jul. 29, 2024, which is based upon and claims priority to Chinese Patent Application No. 202311176126.X, filed on Sep. 12, 2023, the entire contents of which are incorporated herein by reference.

### TECHNICAL FIELD

The present disclosure relates to a human sebaceous gland carcinoma cell line and use thereof, and belongs to the technical field of cell lines.

### BACKGROUND

Eyelid sebaceous gland carcinoma is the second most common malignant eyelid tumor, accounting for nearly 40% of all malignant eyelid tumors in China. Patients with T4-stage eyelid sebaceous gland carcinoma account for 6.25% of all patients with eyelid sebaceous gland carcinoma. Patients with T4-stage eyelid sebaceous gland carcinoma have a 4-fold higher risk of metastasis and approximately 15-fold higher risk of disease-specific mortality than patients with early-stage eyelid sebaceous gland carcinoma. It is extremely urgent to find targeted drug therapies for highly invasive and poorly differentiated eyelid sebaceous gland carcinoma. However, the etiology and pathogenesis of eyelid sebaceous gland carcinoma remain unclear. The establishment of human eyelid sebaceous gland carcinoma cell lines provides abundant experimental materials for understanding the biological characteristics of tumors and studying the carcinogenesis, molecular genetics, and metastatic evolution mechanisms of tumors, and can contribute to studies on tumor mechanisms and chemotherapy resistance mechanisms.

Currently, the most common mutations in eyelid sebaceous gland carcinoma occur in TP53, and approximately 70% of patients with eyelid sebaceous gland carcinoma undergo TP53 mutations. TP53 is a classic tumor suppressor gene. Mutations of TP53 play a key role in the occurrence and development of many tumors. There are no TP53 mutations in the existing sebaceous gland carcinoma cell lines. Therefore, it is essential to establish new sebaceous gland carcinoma cell lines with TP53 mutations to enrich and complete the sebaceous gland carcinoma cell line repository and provide suitable in vitro models for studying tumor heterogeneity, thereby promoting both basic and clinical research on sebaceous gland carcinoma.

### SUMMARY

An objective of the present disclosure: In view of the issue that the existing human sebaceous gland carcinoma cell lines demonstrate low biodiversity and fail to meet research needs in recent years, the present disclosure provides a novel human sebaceous gland carcinoma cell line and a use thereof. This human sebaceous gland carcinoma cell line is classified differently from the existing human sebaceous gland carcinoma cell lines. This human sebaceous gland carcinoma cell line carries a TP53 mutation, and is more in line with clinically prevalent tumor types. This human sebaceous gland carcinoma cell line enables stable passage, excellent tumorigenicity, and large-scale expansion and long-term in vitro subculturing. Thus, this human sebaceous gland carcinoma cell line is suitable for constructing animal models.

To achieve the above objective, the present disclosure adopts the following technical solutions: The present disclosure provides a human sebaceous gland carcinoma cell line, where the human sebaceous gland carcinoma cell line was deposited in the China Center for Type Culture Collection (CCTCC) on Aug. 31, 2023, with an accession number CCTCC NO: C2023113.

The present disclosure also provides a use of the human sebaceous gland carcinoma cell line in preparation of a human sebaceous gland carcinoma animal model.

The present disclosure also provides a use of the human sebaceous gland carcinoma cell line in screening anti-sebaceous gland carcinoma drugs.

The present disclosure also provides a use of the human sebaceous gland carcinoma cell line in studying mechanisms of occurrence, development, and metastasis of human sebaceous gland carcinoma and screening biomarkers for the human sebaceous gland carcinoma.

Compared with the prior art, the present disclosure has the following beneficial effects:
(1) The human sebaceous gland carcinoma cell line SHNPH-SeC in the present disclosure has been identified as a novel single cell line by short tandem repeat (STR) genotyping. This human sebaceous gland carcinoma cell line exhibits strong adherence and stable cellular characteristics, can be stably passaged for multiple generations, and possesses rapid proliferation and migration capabilities, which are in line with the characteristics of malignant tumor cells.
(2) The human sebaceous gland carcinoma cell line SHNPH-SeC in the present disclosure carries a TP53 mutation, and is distinctly different from the sebaceous gland carcinoma cell lines (which are TP53-negative) in the current cell repository. TP53 is the most common mutated gene in human sebaceous gland carcinoma, and over 60% of patients with sebaceous gland carcinoma have a TP53 mutation. Thus, the present disclosure provides a novel experimental material with close biological characteristics to clinical tumors for human sebaceous gland carcinoma research, which further enriches the cell repository.
(3) Compared with the prior art, the human sebaceous gland carcinoma cell line SHNPH-SeC of the present disclosure exhibits high sensitivity to chemotherapy and can serve as a research cell line for effective drug screening. The human sebaceous gland carcinoma cell line of the present disclosure demonstrates a strong tumor-forming ability in vitro, and enables the successful establishment of a sebaceous gland carcinoma model for basic research and drug screening. When the human sebaceous gland carcinoma cell line of the present disclosure is inoculated for tumor formation, a tumor volume can reach 467 mm³ on day 21 after inoculation.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1D show optical morphology images of SHNPH-SeC cells at P0 (FIG. 1A), P5 (FIG. 1B), P30 (FIG. 1C), and P80 (FIG. 1D);

FIG. 2 shows karyotyping and STR genotyping results for SHNPH-SeC cells;

FIGS. 3A-3B show proliferative capacity results of SHNPH-SeC cells detected by Cell Counting Kit-8 (CCK-8) assay and invasion-migration ability results of SHNPH-SeC cells detected by Transwell invasion assay, where FIG. 3A shows a growth curve of SHNPH-SeC cells plotted by the CCK-8 assay and FIG. 3B shows a crystal violet staining image of the Transwell invasion assay;

FIG. 4 is a comparison diagram of immunohistochemical staining results for a clinical surgical tissue sample of human sebaceous gland carcinoma and immunofluorescence staining results for SHNPH-SeC cells;

FIG. 5 shows a tumor growth curve after xenotransplantation of SHNPH-SeC cells into mice;

FIGS. 6A-6B show Hematoxylin and Eosin (H&E) staining results of a primary tumor tissue (FIG. 6A) and a tumor tissue generated from SHNPH-SeC cells in nude mice (FIG. 6B);

FIG. 7 shows immunofluorescence assay results of a tumor tissue generated from SHNPH-SeC cells in nude mice;

FIG. 8 shows mutation signature analysis results for the SHNPH-SeC and the primary tumor; and

FIGS. 9A-9B show drug sensitivity test results of SHNPH-SeC cells to different clinical drugs against eyelid sebaceous gland carcinoma, where FIG. 9A shows drug sensitivity test results for mitomycin C (MMC) and FIG. 9B shows drug sensitivity test results for 5-fluorouracil (5-FU).

### DEPOSITION INFORMATION

The human sebaceous gland carcinoma cell line SHNPH-SeC was deposited in the China Center for Type Culture Collection (CCTCC) at Wuhan University, Wuhan, China on Aug. 31, 2023, with an accession number CCTCC NO: C2023113.

### DETAILED DESCRIPTION OF THE EMBODIMENTS

In order to make the present disclosure easily understandable, preferred examples are provided to describe the present disclosure in detail below with reference to the accompanying drawings.

Unless otherwise specified, the test methods used in the following examples are conventional methods, and the materials and reagents used in the following examples are commercially available reagents and materials.

### Example 1 Preparation of a Human Sebaceous Gland Carcinoma Cell Line (Cell Line SHNPH-SeC)

### 1.1 Sample Source

On Aug. 31, 2021, a post-surgical sample was collected from an 87-year-old patient with stage T4a sebaceous gland carcinoma at the Shanghai Ninth People's Hospital affiliated to Shanghai Jiao Tong University School of Medicine. The chief complaint of the patient was as follows: There had been a mass in the medial canthus of the left eye for 2 years. The mass rapidly grew over the past 6 months, such that the overall eyelid was swollen and the eyeball underwent exotropia and could not rotate. The patient was pathologically diagnosed with sebaceous gland carcinoma. The sample had a size of 4 cm*2.5 cm*2.2 cm. An informed consent form was signed by the patient prior to the surgery.

### 1.2 Sample Treatment

The fresh sebaceous gland carcinoma sample resulting from surgical excision was rinsed three times with phosphate buffered saline (PBS). A tumor tissue was separated from a normal tissue.

Under sterile conditions, the tumor tissue was cut into small tissue fragments and soaked in an RPMI-1640 medium (purchased from Gibco) (including 2% of fetal bovine serum, 200 mg/mL of streptomycin, and 200 IU/mL of penicillin) at room temperature.

Under sterile conditions, the small tissue fragments were further cut into tissue fragments of approximately 1 mm*1 mm using a surgical scalpel, and then placed in a 15 mL sterile centrifuge tube. RPMI-1640 including 0.5% of collagenase I (purchased from Invitrogen) was added, and digestion was conducted for 4 h at 37° C. under shaking.

A resulting digestion system was centrifuged for 4 min at 800 rpm and room temperature. A treatment was conducted for 2 min with trypsin-ethylenediamine tetraacetic acid (EDTA) (including 0.53 mM of EDTA and 0.05% of trypsin, purchased from Gibco). Then, neutralization and precipitation were conducted in an RPMI-1640 medium (including 10% of fetal bovine serum, 100 mg/mL of streptomycin, and 100 IU/mL of penicillin). Centrifugation was conducted. Cells were collected and cultured in an incubator at 37° C. and 5% CO₂ for two weeks with an RPMI-1640 medium (including 10% of fetal bovine serum, 100 mg/mL of streptomycin, and 100 IU/mL of penicillin).

### 1.3 Monoclonal Purification

Cells were collected from a region with high cell viability and prepared into a single-cell suspension at a concentration of 10 cells/mL. Then, 100 μL of the single-cell suspension was added to each well of a 96-well plate and cultured to create single-cell proliferation wells for monoclonal purification.

Resulting single-cell clones were digested with trypsin for 1 min, and then expanded using an RPMI-1640 medium in a 37° C. and 5% CO₂ incubator. Passaging was conducted once a week. Cells exhibited a robust growth state and a relatively uniform morphology, and had been successfully passaged for 80 or more generations.

The human sebaceous gland carcinoma cells were named a human sebaceous gland carcinoma cell line SHNPH-SeC, which was deposited in the China Center for Type Culture Collection (CCTCC) on Aug. 31, 2023, with an accession number CCTCC NO: C2023113.

### Example 2 Biological Characteristics and Use of the Human Sebaceous Gland Carcinoma Cell Line SHNPH-SeC

### 2.1 Morphological Observation for the Human Sebaceous Gland Carcinoma Cell Line SHNPH-SeC

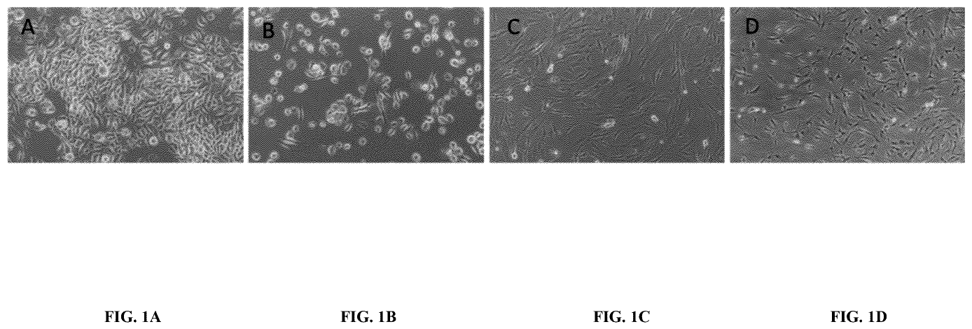

SHNPH-SeC cells subcultured were collected and observed under an optical microscope (100×) for growth of live cells. Results were shown in FIG. 1A to FIG. 1D. FIG. 1A to FIG. 1D show optical morphology images of SHNPH-SeC cells at P0, P5, P30, and P80, respectively. As shown in FIG. 1A to FIG. 1D, the SHNPH-SeC cells grow vigorously and adherently with a clear background and few impurities, and the adherent cells are spindle-shaped.

### 2.2 STR Analysis for a Genetic Material in the Sebaceous Gland Carcinoma Cell Line SHNPH-SeC

STRs, also known as microsatellite DNAs, refer to tandemly repeated DNA sequences with few base pairs (2 to 6 base pairs) as a core unit on a chromosome (a repeat count is 10 to 60, and a gene fragment includes 400 or less base pairs). The number of repetitions of each core unit varies among individuals, resulting in alleles with different lengths. Therefore, a repeat count in a set of STR sequences is nearly unique to each individual, and can serve as a genetic identity characteristic for an individual. The repeat count can also used as the primary method for identifying an identity and origin of a cell in cell biology.

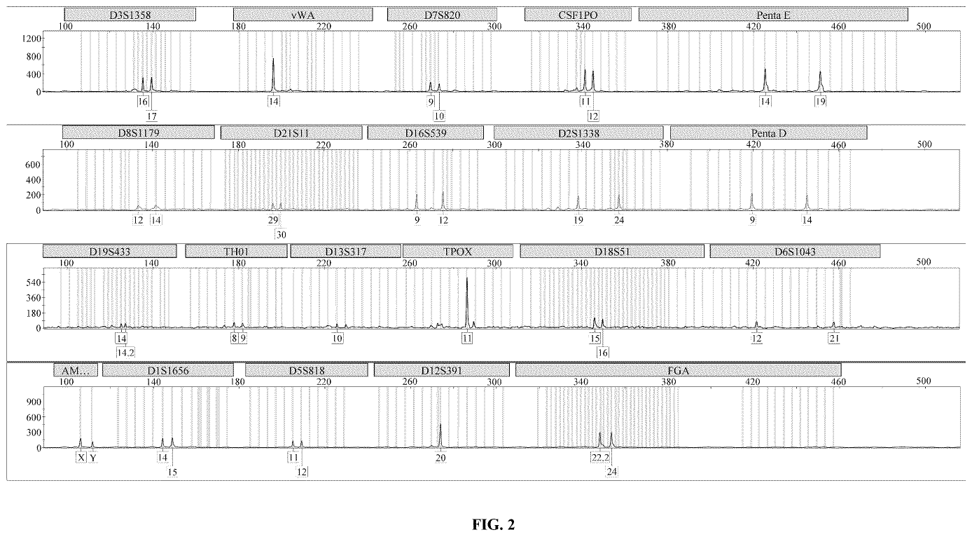

Genomic DNA was extracted from cells using the QIAGEN genomic extraction kit (QIAamp PowerFecal DNA Kit purchased from QIAGEN), and 21 STR loci and a gender gene Amelogenin were detected on an ABI 3730XL genetic analyzer. Detection results were shown in FIG. 2.

The STR sequence search was conducted in the Deutsche Sammlung von Mikroorganismen und zellkulturen (DSMZ) database, the American Type Culture Collection (ATCC) database, the Japanese Collection of Research Bioresources (JCRB) database, and the RIKEN Institute of Kagaku Kenkyū (RIKEN) database. No identical STR results were found. No loci matching this cell line were found, indicating that this cell line is a novel cell line. No multiallelic loci were found, indicating that this cell line includes a single cell and is free from contamination by other cells.

### 2.3 Studies on Growth Kinetics and Invasiveness of the Sebaceous Gland Carcinoma Cell Line SHNPH-SeC

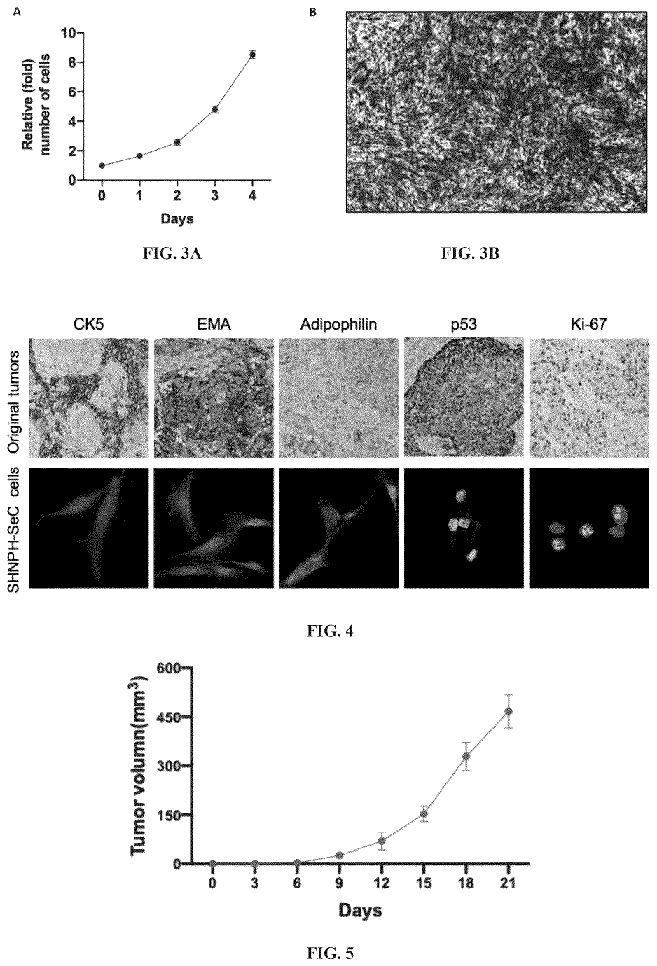

Cells in a logarithmic growth phase were harvested and prepared into a cell suspension at a concentration of 2×10⁴ cells/mL. The cell suspension was inoculated into a 96-well plate at 100 μL/well. The cell proliferation in the 96-well plate was measured by the CCK-8 method on day 0, day 1, day 2, day 3, and day 4. The CCK-8 method was performed according to instructions of a kit. Specifically, a sample was incubated with 10 μL of a CCK-8 solution for 2 h in a 37° C. and 5% CO₂ incubator, and an OD value was then measured at 450 nm. A growth curve of the sebaceous gland carcinoma cell line SHNPH-SeC was shown in FIGS. 3A-3B. As shown in the growth curve of FIG. 3A, the sebaceous gland carcinoma cell line SHNPH-SeC required a doubling time of approximately 36 h, indicating a strong proliferative capacity.

An invasion assay was conducted to evaluate a migration ability of the cell line. A Transwell chamber was placed in a culture plate. An upper chamber was filled with a medium including 2% of PBS, and a lower chamber was filled with 900 μL of a complete medium. The upper and lower chambers were separated by a polycarbonate membrane (with 8 μm pores). 5×10⁴ cells were added to the upper chamber, incubated for 24 h, and then stained with 0.25% crystal violet. An image illustrating the migration of cells to the lower chamber was acquired under a microscope (as shown in FIG. 3B). According to the results, the majority of cells migrated through the membrane into the lower chamber within 24 h, indicating that the sebaceous gland carcinoma cell line SHNPH-SeC exhibited an invasive behavior.

### 2.4 Determination of Biological Characteristics of the Human Sebaceous Gland Carcinoma Cell Line SHNPH-SeC

2.4.1 SHNPH-SeC cells and a clinical surgical tissue sample of human sebaceous gland carcinoma were subjected to immunofluorescence staining and immunohistochemical staining, respectively, according to the following steps:

Treating procedure for the SHNPH-SeC cells: The cells were inoculated into a 24-well plate, cultured for 24 h, then fixed with 4% paraformaldehyde for 30 min, treated with 0.5% Triton X-100 for 15 min, blocked with 5% goat serum (purchased from Invitrogen) for 1 h, incubated with a primary antibody (anti-CK5, anti-EMA, anti-Adipophilin, anti-p53, and anti-Ki-67 antibodies) overnight at 4° C., and then incubated with a secondary antibody at room temperature for 1 h.

Treating procedure for the clinical surgical tissue sample of human sebaceous gland carcinoma: The clinical surgical tissue sample was cut to approximately 1 cm×1 cm×0.3 cm, placed in a tissue embedding cassette, fixed with 10% neutral buffered formalin, dehydrated with 75% ethanol, 80% ethanol, 95% ethanol, 95% ethanol, 100% ethanol, and 100% ethanol successively for 2 h in each, then treated twice with xylene for 30 min each time, immersed in paraffin at 60° C. for 2 h, embedded, and sectioned to produce sections each with a thickness of 4 μm. The sections were baked in a 60° C. oven for 1 h, then dewaxed with xylene, then treated with 0.1% Triton X-100 and 0.3% H₂O₂, blocked with 4% goat serum, and incubated with a primary antibody (anti-CK5, anti-EMA, anti-Adipophilin, anti-p53, and anti-Ki-67 antibodies) at room temperature for 60 min.

2.4.2 Microscopic imaging was performed using an Eclipse microscope from Nikon in Japan to comparatively observe the staining conditions of biomarkers in the clinical surgical tissue sample of human sebaceous gland carcinoma and the SHNPH-SeC cells. Immunofluorescence staining results indicated the positive expression of CK5, EMA, Adipophilin, p53, and Ki-67 in the SHNPH-SeC cells with a labeling index of 60%. The immunofluorescence staining results were consistent with immunohistochemical staining results for the clinical surgical tissue sample, as shown in FIG. 4.

### 2.5 Tumorigenicity of Cells

SHNPH-SeC cells in a logarithmic growth phase were collected and prepared into a cell suspension. 1×10⁶ cells were unilaterally injected into a subcutaneous tissue on the right flank of male NOD/SCID mice. Tumor formation was monitored and recorded, and a tumor volume was assessed every three days. A tumor growth curve was plotted. A tumor was collected on day 21 after implantation.

A tumor growth curve from day 0 to day 21 after implantation was shown in FIG. 5. It was observed that, from day 0 to day 21, a tumor diameter increased rapidly, and a tumor volume reached 467 mm³ on day 21. It could be seen that SHNPH-SeC cells exhibited a very strong tumor-forming ability in vitro.

### 2.6 Pathological Identification of a Tumor

A tumor formed on day 21 after the SHNPH-SeC cells were subcutaneously inoculated into nude mice as described in the step 2.5 was subjected to paraffin embedding, sectioning, and H&E staining according to the following steps: The tumor was cut to approximately 1 cm×1 cm×0.3 cm, placed in a tissue embedding cassette, fixed with 10% neutral buffered formalin, dehydrated with 75% ethanol, 80% ethanol, 95% ethanol, 95% ethanol, 100% ethanol, and 100% ethanol successively for 2 h in each, permeabilized with xylene twice for 30 min each time, immersed in paraffin at 60° C. for 2 h, embedded using a Leica JUNG embedding machine of Germany, and sectioned by a Leica 2135 microtome of Germany to produce sections each with a thickness of 4 μm. The sections were baked in a 60° C. oven for 1 h, stained using a Leica ST5020 stainer of Germany, and mounted using a Leica CV5030 coverslipper of Germany.

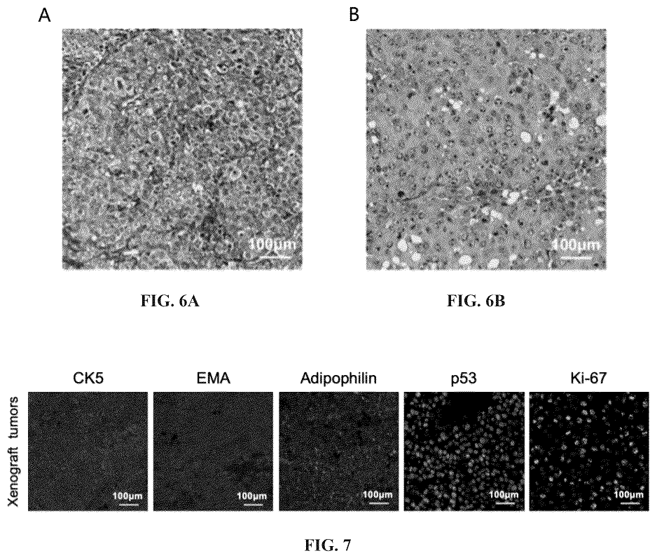

Microscopic imaging was performed under an optical microscope to observe an H&E staining result of tumor cells generated from the SHNPH-SeC cells in nude mice. The result was shown in FIG. 6B. FIG. 6A shows an H&E staining image of a primary tumor. It can be observed that the SeC xenograft in mice demonstrates consistent histological characteristics with the primary tumor.

### 2.7 Immunofluorescence Assay of a Tumor Tissue in Nude Mice

The tumor tissue was collected from the nude mice and subjected to immunofluorescence assay (an experimental procedure was the same as the immunofluorescence assay method described in section 2.4). Immunofluorescence assay results of the tumor tissue were shown in FIG. 7. It can be observed that immunofluorescence staining results of the SeC xenograft are the same as the results of the primary tumor and the SHNPH-SeC cells, and the SeC xenograft is also positive for CK5, EMA, Adipophilin, p53, and Ki-67 (FIG. 4).

### 2.8 Mutation Analysis of SHNPH-SeC Cells

Whole exome sequencing can comprehensively reflect the genetic information of a biological sample and reveal the genomic mutation characteristics. Thus, whole exome sequencing is a common approach for determining whether there is a gene mutation. Genomic DNA was extracted from a primary tumor tissue and SHNPH-SeC cells using a DNA extraction kit (purchased from Beyotime). DNA was subjected to quantification and integrity analysis with a spectrophotometer of Thermo Fisher Scientific in the United States and 1% agarose gel electrophoresis, respectively. Genomic DNA was captured with the Agilent SureSelect Human All Exon V6 library according to instructions, and sequenced on the HiSeq X10 sequencing platform. Moreover, all single nucleotide polymorphisms, insertions and deletions, copy number variations, and structural variations were analyzed.

Mutation signature analysis reveals that there are consistent characteristics between the SHNPH-SeC cells and the primary tumor, as shown in FIG. 8. In the Catalogue Of Somatic Mutations In Cancer (COSMIC) database, the top ten genes associated with sebaceous gland carcinoma are TP53, RB1, CTNNB1, LEF1, ERBB2, KMT2C, PIK3CA, PBRM1, KMT2D, and IL6ST, respectively. A mutation rate of TP53 is approximately 70%. The SHNPH-SeC cells and the primary tumor share seven protein-altering mutations, all of which are related to the top ten genes associated with sebaceous gland carcinoma. TP53, the most frequently mutated gene in sebaceous gland carcinoma, includes a missense mutation (c.C215G: p.P72R), as shown in Table 1 below:

TABLE 1

| Chr | Start | Ref | Alt | Gene | Amino Acid Change |
|---|---|---|---|---|---|
| chr7 | 151927021 | C | A | KMT2C | p. C988F |
| chr7 | 151945007 | C | T | KMT2C | p. G838S |
| chr7 | 151945204 | G | A | KMT2C | p. S772L |
| chr7 | 151970856 | T | A | KMT2C | p. T316S |
| chr7 | 151970931 | G | A | KMT2C | p. L291F |
| chr17 | 7579472 | G | C | TP53 | p. P72R |
| chr17 | 37884037 | C | G | ERBB2 | p. P824A |

According to existing technical records, all sebaceous gland carcinoma cell lines available in the current cell repository do not have a TP53 mutation. In contrast, the sebaceous gland carcinoma cell line SHNPH-SeC of the present disclosure carries a TP53 mutation. It can be known that the sebaceous gland carcinoma cell line SHNPH-SeC of the present disclosure is distinct from the cell lines available in the current cell repository. Since approximately 70% of sebaceous gland carcinoma cases are characterized by TP53 mutations, the present disclosure can well enrich the sebaceous gland carcinoma cell repository and further advance the development of fundamental experiments.

### 2.9 Drug Sensitivity Tests of the Sebaceous Gland Carcinoma Cell Line SHNPH-SeC

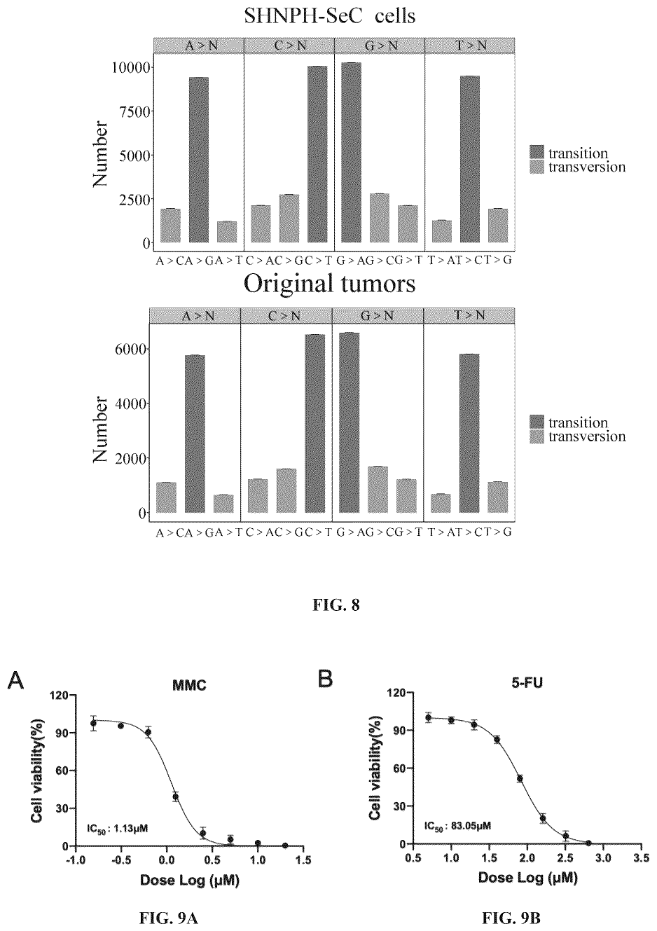

Cells in a logarithmic growth phase were collected and prepared into a cell suspension with a concentration of 2×10⁴ cells/mL. The cell suspension was inoculated into a 96-well plate at 100 μL/well. When a cell density reached approximately 80%, MMC was added to each well at concentrations of 0 μmol/L, 0.15625 μmol/L, 0.3125 μmol/L, 0.625 μmol/L, 1.25 μmol/L, 2.5 μmol/L, 5 μmol/L, 10 μmol/L, and 20 μmol/L or 5-FU was added to each well at concentrations of 0 μmol/L, 5 μmol/L, 10 μmol/L, 20 μmol/L, 40 μmol/L, 80 μmol/L, 160 μmol/L, 320 μmol/L, and 640 μmol/L. 48 h later, a cell viability in each well was detected by the CCK-8 method, with a specific process as described in section 2.3. Results of drug sensitivity tests of MMC and 5-FU are shown in FIG. 9A and FIG. 9B, respectively. Half-maximal effective concentration (EC50) values of MMC and 5-FU in the sebaceous gland carcinoma cell line SHNPH-SeC are 1.13 μM and 83.05 μM, respectively. It has been previously reported that EC50 values of MMC and 5-FU in the sebaceous gland carcinoma cell line BP50 without a TP53 mutation are 10.4 μM and 80 mM, respectively. It can be seen that the sebaceous gland carcinoma cell line SHNPH-SeC is more sensitive to both MMC and 5-FU than the sebaceous gland carcinoma cell line BP50.

Based on the above experimental observations and verifications, SHNPH-SeC cells growing in vitro exhibit strong adherence and stable cellular characteristics, can be stably passaged for multiple generations, and undergo malignant growth. This human sebaceous gland carcinoma cell line has been identified as a novel single cell line by STR genotyping. Since this cell line is positive for sensitive and reliable biomarkers of eyelid sebaceous gland carcinoma, including CK5, EMA, Adipophilin, and p53, this cell line is identified as a sebaceous gland carcinoma cell line. SHNPH-SeC cells can be extensively expanded, show high proliferative activity, and enable tumor formation in nude mice. The consistent staining results of a primary tumor, SHNPH-SeC cells, and a xenograft tumor indicate that SHNPH-SeC cells are derived from a patient with eyelid sebaceous gland carcinoma. The similar H&E staining results of the primary tumor and the xenograft tumor further indicate that SHNPH-SeC is an eyelid sebaceous gland carcinoma cell line. It can be further confirmed that there are identical mutation characteristics between the primary tumor and the SHNPH-SeC cells, and the SHNPH-SeC cells carry a TP53 mutation most frequently observed in sebaceous gland carcinoma. Regarding malignant characteristics, the sebaceous gland carcinoma cell line SHNPH-SeC possesses characteristics such as high Ki-67 positive rate, abundant mutations in sebaceous gland carcinoma-associated genes, rapid proliferation and migration in vitro, and tumorigenesis in vivo. The drug sensitivity characteristics also prove that SHNPH-SeC cells respond well to chemotherapy, even with a better response than a sebaceous gland carcinoma cell line free of a TP53 mutation. The human sebaceous gland carcinoma cell line SHNPH-SeC, a nude mouse in which a tumor is formed from this human sebaceous gland carcinoma cell line, and a clinical human eyelid sebaceous gland carcinoma tissue sample from which this human sebaceous gland carcinoma cell line is derived collectively constitute a correlated research system. This correlated research system provides new experimental materials for investigating the correlation of anti-cancer drug sensitivity and resistance among in vitro, in vivo, and clinical practice and for studying the occurrence, development, metastasis, and biomarkers of human sebaceous gland carcinoma.

The above examples are merely preferred examples of the present disclosure rather than limitations to the present disclosure in any form and essence. It should be noted that those of ordinary skill in the art may make various improvements and supplementations without departing from the present disclosure, and these improvements and supplementations should be regarded as falling within the protection scope of the present disclosure.

What is claimed is:

1. A human sebaceous gland carcinoma cell line, wherein the human sebaceous gland carcinoma cell line was deposited in the China Center for Type Culture Collection (CCTCC) on Aug. 31, 2023, with an accession number CCTCC NO: C2023113.

2. A method for preparing a human sebaceous gland carcinoma animal model, comprising using the human sebaceous gland carcinoma cell line according to claim 1.

3. A method for screening anti-sebaceous gland carcinoma drugs, comprising using the human sebaceous gland carcinoma cell line according to claim 1.

4. A method for studying mechanisms of occurrence, development, and metastasis of human sebaceous gland carcinoma and screening biomarkers for the human sebaceous gland carcinoma, comprising using the human sebaceous gland carcinoma cell line according to claim 1.
